(19) United States
(12) Patent Application Publication
(10) Pub. No.: US 2005/0079627 A1
(43) Pub. Date: Apr. 14, 2005

(54) Absorbance monitoring

(76) Inventors: Gethin Rhys Jones, Bridgend (GB); David Venn Ludlow, Bridgend (GB)

Correspondence Address: FULWIDER PATTON LEE & UTECHT, LLP;HOWARD HUGHES CENTER, 6060 CENTER DRIVE, TENTH FLOOR, LOS ANGELES, CA, 90045, US

(21) Appl. No.: 10/496,026

(22) PCT Filed: Nov. 20, 2002
(86) PCT No.: PCT/GB02/05397

(30) Foreign Application Priority Data
Nov. 20, 2001 (GB) 0127861.3

(51) Int. Cl. G01N 33/00

(52) U.S. Cl. 436/164; 422/68.1

(57) ABSTRACT

A method for monitoring the absorbance of a sample, which method includes: directing a first beam of light having a first wavelength along an optical path through a portion of the sample and measuring the absorbance of the first wavelength so as to obtain a reference absorbance; directing a second beam of light having a second wavelength substantially along the optical path and measuring the absorbance of the sample so as to obtain a measured sample absorbance; and analysing the reference absorbance and the measured sample absorbance to obtain a true sample absorbance.

The present invention is concerned with a method and apparatus suitable for use in monitoring the absorbance of a sample. The present invention extends to a method and apparatus suitable for use in detection of an analyte by measuring a chromogen formed by a chemical reaction between an analyte and a reagent.

Many chemical detection techniques rely on the formation of a chromogen that is visible. Such chemical detection techniques have a very wide range of applications. It is also possible to detect the presence of biological materials or micro-organisms by the formation of coloured end products of enzymic reactions. One such application of a chemical chromogenic reaction is the use of the detection of protein using copper sulphate and BCA, which is used in a product sold by Biotrace Ltd under the trade mark PRO-TECT.

The PRO-TECT device is activated when the analyte being detected (which has been for example, arranged or collected on a swab-bud or the like) is introduced to a reagent. The chemical reaction between the analyte and the reagent results in the reagent changing colour, typically from green to grey or purple (dependent, of course on the reagent and the analyte being used). When using the PRO-TECT apparatus if the reagent remains green this is considered to be an analyte negative result, thereby deeming the swab-bud (and therefore the surface swabbed by the bud) clean, that is substantially free from the analyte being detected. However, if the reagent turns grey, the surface swabbed by the swab-bud is classed as a caution, whereas if the reagent turns purple, the surface swabbed by the swab-bud is considered as a failure (i.e. the surface is contaminated with the analyte).

Whilst such methods and apparatus provide a guide as to whether the analyte being detected is present, it relies on the colour change being visually assessed by the technician carrying out the test.

In order to alleviate operator error, measuring the colour change using a detector and a light source (the light source emitting light having a predetermined wave length) has been used. The light passes through a vial containing a sample and the absorbance is measured by the detector. A calculation of the colour can then be made.

In many other calorimetric tests the formation of the chromogen can be detected by use of a spectrophotometer in the absorbance mode. The wavelength of the light emitted is modified to that suitable for detection of the particular chromogen by using filters. These are usually narrow band optical filters to generate the required wavelength(s) from a wide bandwidth light source (for example a lamp). The filters are mechanically selectable, either manually with levers or automatically by the instrument under electromechanical control.

However, the standard technique when applied in the case of the PRO-TECT test has a number of disadvantages. These disadvantages include the final resting position of a swab-bud which has been immersed in a vessel containing the reagent is not considered. The final resting position of the swab-bud must be considered as stray light from the source may reflect off the bottom of the bud and into the detector.

In addition, the actual physical properties of the vessel, such as it thickness have not been considered in previous methods.

The vessel physical properties such as the thickness and also mould tool cavity to cavity may vary as the vessels are typically moulded using eight cavity tooling, therefore, no two cavities are identical and as such produce slightly different sample vessels. Furthermore, in some cavities, the wall thickness in the optimum viewing area may vary by up to about 30%. As the light shines through the sample vessel, the sample and the vessel act as a lens, the optical properties of which are affected by wall thickness etc.

It is therefore an aim of the present invention to alleviate at least some of the disadvantages identified above.

It is a further aim of the present invention to provide a method and apparatus for monitoring the absorbance of a sample.

It is yet a further aim of the present invention to provide apparatus suitable for use in monitoring colour change in a sample.

It is still yet a further aim of the present invention to provide a method of monitoring colour change in a sample. Therefore, according to a first aspect of the present invention there is provided a method for monitoring the absorbance of a sample, which method includes:
directing a first beam of light having a first wavelength along an optical path through a portion of the sample and measuring the absorbance of the first wavelength so as to obtain a reference absorbance;
directing a second beam of light having a second wavelength substantially along the optical path and measuring the absorbance of the sample so as to obtain a measured sample absorbance; and
analysing the reference absorbance and the measured sample absorbance to obtain a true sample absorbance.

The first wavelength is preferably selected at a wavelength whereby the sample has a substantially similar absorbance irrespective of the colour of the sample.

The second wavelength is preferably selected at a wavelength whereby the sample has a peak absorbance irrespective of the colour of the sample. Peak absorbance is substantially at a wavelength where the absorbance varies the greatest according to colour change.

Advantageously, the true sample absorbance is substantially not affected by external factors such as the thickness of the vessel having the sample therein or reflected light, as both the reference absorbance and the measured sample absorbance will be subject to substantially the same external factors. Therefore variables will be cancelled out in the true sample absorbance.

The analyte may include (but is not limited to) the product of a chemical or biochemical reaction, biological material, micro-organisms, proteins, bacteria, or the like.

The method is particularly advantageous for measuring the absorbance of a sample which has undergone a chemical or biochemical reaction. In particular reactions whereby the starting reagent has a different absorbance to the final end product. The method may advantageously monitor the absorbance of a chromogen formed as a result of the chemical reaction.

Preferably, the first wavelength and the second wavelength are directed through light directing means (such as an optical fibre) prior to passing through the sample. Advantageously, the light directing means ensures that each wavelength passes through the sample along substantially the same optical path. Therefore, physical factors affecting the first wavelength will also affect the second wavelength. The light directing means also serves as a light combining means.

Desirably, the first wavelength and the second wavelength pass through a lens (such as a sapphire ball lens) prior to passing through the sample so that a narrow, substantially parallel beam is produced.

According to a preferred embodiment of the present invention, the method is suitable for measuring the colour of a sample, where each colour has a different absorbance. Preferably, the sample undergoes a colour change during a chemical reaction; the method according to the invention is typically carried out after the chemical reaction. The chemical reaction may be a reaction which occurs due to the reaction of a reagent with a protein.

Therefore, according to a further aspect of the present invention, there is provided a method of monitoring the presence of an analyte on a surface, which method includes:
permitting the surface to contact a reagent so that analyte present on the surface may (chemically) react with the reagent if the analyte is present;
directing a first wavelength along an optical path through a portion of the reagent and measuring the absorbance of the sample so as to obtain a reference absorbance;
directing a second beam of light having a second wavelength substantially along the optical path and measuring the absorbance so as to obtain a measured sample absorbance;
and analysing the reference absorbance and the measured sample absorbance to obtain a true sample absorbance which provides an indication the amount of analyte present on the sample which has reacted with the reagent.

The indication of the amount of analyte present may be presented in relative colour units.

According to a particularly preferred embodiment of this aspect of the present invention, the analyte is a protein and the reagent comprises a mixture of BCA and copper sulphate. The reagent is typically a mixture of A(1% disodium-2,2′-bisinchonate, 2% Na₂CO₃.H₂O, 0.16% sodium tartrate, 0.4% NaOH, 0.95% NaHCO₃ and 95.49% water) and B (deionised aquesus solution containing 4% by weight cupric sulfate) preferably in the ratio 100:2. In this embodiment, if no analyte is present the reagent remains green, however, if analyte is present the reagent changes colour from green to grey or purple depending upon the amount of analyte present. The absorbance of a sample varies with the colour of the sample. The method preferably is used to measure the chromogen formed in the reaction.

Preferably, the first wavelength is set at or above 750 nm in the infra red region where the absorbance is similar for purple, grey and green. Further preferably, the first wavelength is at or about 850 mm.

Preferably, the second wavelength is set at or about 562 nm in the green region (where the peak absorbance occurs). According to a particularly preferred embodiment of the first aspect of the present invention, there is provided a method of monitoring the absorbance of a sample, which method includes directing a first beam of infra red light along a path length through the sample and measuring the absorbance so as to obtain a reference absorbance;
directing a second beam of green light along the path length through the sample and measuring the absorbance of the second beam in the sample so as to obtain a measured sample absorbance; and
analysing the reference absorbance and the measured sample absorbance so as to obtain a true sample absorbance.

According to a further aspect of the present invention, there is provided apparatus for monitoring the absorbance of a sample, which apparatus includes:
a first light source arranged to emit light at a first wavelength through the sample along an optical path;
a second light source arranged to emit light at a second wavelength through the sample substantially along the optical path; and
a detector arranged to measure the absorbance of the light emitted from the first light source and the second light source which has passed through the sample.

According to a particularly preferred embodiment of the present invention the apparatus further includes light directing means arranged to combine the light from the first light source and the light from the second light source so that the light from both light sources travel substantially along the same path.

Accordingly, there is further provided apparatus for monitoring the absorbance of a sample, which apparatus includes:
a first light source arranged to emit light at a first wavelength through the sample along an optical path;
a second light source arranged to emit light at a second wavelength through the sample substantially along the optical path;
light directing means arranged to combine the light from the first light source and the light from the second light source such that light from both light sources travel substantially along the same path; and
a detector arranged to measure absorbance of light emitted from the first light source and the second light source, which has passed through the sample.

It is particularly preferred that the light directing means is an optical fibre. The optical fibre preferably has a length about 0.5 to 10 mm. The optical fibre is preferably arranged to combine light emitted from the first light source and light emitted from the second light source so that both light sources travel substantially along the same path.

The light emerging from the light directing means may exit at wide angles. It is therefore particularly preferred that the apparatus further includes a lens (preferably a ball lens, such as a sapphire ball lens), arranged to produce a narrow, substantially parallel or near parallel beam of light which will subsequently pass through the sample. Advantageously, the lens minimises scattering of the light and also reduces the occurrence of stray scattering. The lens is typically arranged substantially between the light combing means and the sample.

It is particularly preferred that the apparatus further includes a reflector. The reflector is typically arranged such that light emitted from the first light source and/or the second light source is reflected by the reflector and the reflected light is directed to the light directing means. The use of a reflector has the advantage of permitting the first light source and/or the second light source to be rotated and mounted within the body of the apparatus thereby reducing the space required for the apparatus. The apparatus is therefore advantageously miniaturised through use of the reflector

The first light source and/or the second light source is preferably an LED set to emit light at the first wavelength and/or the second wavelength. The apparatus preferably includes two or more LED's, preferably arranged to emit light at different wavelengths.

Therefore according to a further embodiment of the present invention the apparatus includes two or more light sources (such as LED's), each light source arranged to emit light at different wavelengths.

The present invention extends to a method of monitoring colour change in a sample, particularly as a result of a chemical reaction. The present invention preferably monitors the absorbance of a chromogen formed by the chemical reaction.

Many chemical reactions result in a colour change. An example of the use of such a reaction is in the detection of proteins whereby a reagent capable of forming colour upon reaction with the protein, is contacted with a surface suspected of having protein thereon.

According to a preferred embodiment of the present invention, the apparatus is suitable for use in monitoring the colour change in a chemical reaction.

According to a particularly preferred embodiment of the present invention the method is suitable for use in monitoring the colour change of a mixture of copper sulphate and BCA (preferably a mixture of A 1% disodium-2,2′-bisinchonate, 2% Na₂CO₃.H₂O, 0.16% sodium tartrate, 0.4% NaOH, 0.95% NaHCO₃ and 95.49% water and B deionised aquesus solution containing 4% by weight cupric sulfate preferably in the ratio 100:2), when the mixture is permitted to contact an analyte such as a protein.

It is particularly preferred that the apparatus may be used with the method of detecting protein identified in European Patent Application 96302706.5.

The present invention may be more clearly understood by reference to the accompanying figures which are given by way of example only, wherein:

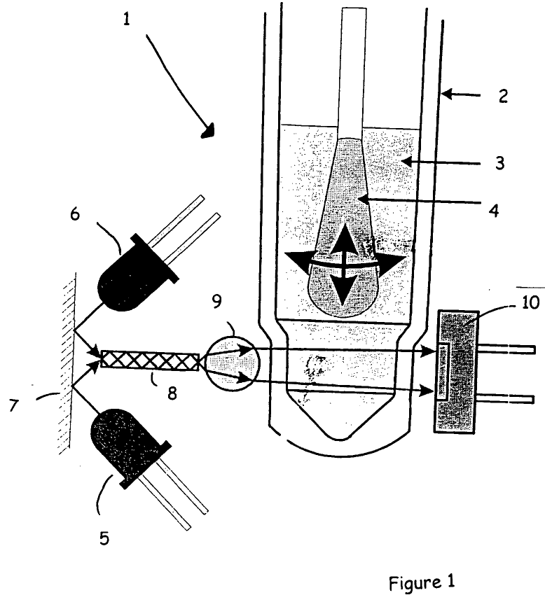

FIG. 1 represents apparatus according to the present invention

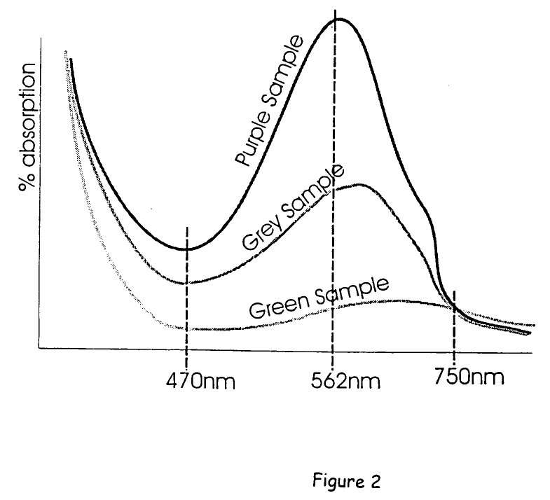

FIG. 2 represents a spectral graph for a green, grey and purple sample.

Referring to FIG. 1, there is provided a monitor for measuring absorbance generally identified by the numeral 1. The sample tube 2 contains reagent 3 which comprises reagents A (1% disodium-2,2′-bisinchonate, 2%. Na₂CO₃.H₂O, 0.16% sodium tartrate, 0.4% NaOH, 0.95% NaHCO₃ and 95.49% water) and B(deionised aqueous solution containing 4% by weight cupric sulfate) in the ratio 100:2.

The swab-bud 4 (which has previously been swabbed over a surface suspected of having protein thereon) is immersed in reagent 3, the protein being permitted to react with the reagent.

The reagent changes colour from green to grey to purple depending on the amount of protein present on the swab (which is a result of the swab being swabbed over a surface having protein thereon).

In use, the swabbed bud 4 is immersed in reagent 3. The first LED which is set to emit infra red light is switched on. The beam of light is reflected off reflector 7 and is directed to optical fibre 8 and to sapphire ball lens 9. The light emerging from the optical fibre 8 which exits a wide angle is transformed into a narrow near parallel beam of light by lens 9. The narrow beam of infra red light passes through the reagent and the absorbance is measured by photodiode 10. The absorbance value is stored as a reference value. The first LED 5 emitting infra red light is switched off.

The second LED 6 emitting green light is switched on and the beam is reflected off reflector 7 and is directed to optical fibre 8 and to sapphire ball lens 9. The beam of green light therefore follows substantially the same path length (through the reagent) as the beam of infra red light. Therefore variations in the final resting position of the swab bud (which may result in light being reflected therefrom) and the thickness and variation of the sample tube 2 will affect both the green light and the infra-red light. A measurement of the absorption is taken by photodiode 10.

The absorbance of the infra-red light and the absorbance of the green light are compared to give a true absorbance value. The true absorbance value may be displayed in ‘Relative Colour Units’ (RCU).

1. A method for monitoring the absorbance of a sample, which method includes:
directing a first beam of light having a first wavelength along an optical path through a portion of the sample and measuring the absorbance of the first wavelength so as to obtain a reference absorbance;
directing a second beam of light having a second wavelength substantially along the optical path and measuring the absorbance of the sample so as to obtain a measured sample absorbance; and
analysing the reference absorbance and the measured sample absorbance to obtain a true sample absorbance.

2. A method according to claim 1, wherein the first wavelength is preferably selected at a wavelength whereby the sample has a substantially similar absorbance irrespective of the colour of the sample.

3. A method according to claim 1 or 2, wherein the second wavelength is preferably selected at a wavelength whereby the sample has a peak absorbance irrespective of the colour of the sample.

4. A method according to any preceding claim, wherein the analyte may include (but is not limited to) the product of a chemical or biochemical reaction, biological material, micro-organisms, proteins, bacteria, or the like.

5. A method according to any preceding claim, which is suitable for measuring the absorbance of a sample which has undergone a chemical or biochemical reaction of a starting reagent with the analyte so as to produce an end product.

6. A method according to claim 5, wherein the starting reagent has a different absorbance to the final end product.

7. A method according to claim 5 or 6, wherein the method monitors the absorbance of a chromogen formed as a result of the chemical reaction.

8. A method according to any preceding claim, wherein the first wavelength and the second wavelength are directed through light directing means prior to passing through the sample.

9. A method according to claim 8, wherein the light directing means is an optical fibre.

10. A method according to any preceding claim, wherein the first wavelength and the second wavelength pass through a lens prior to passing through the sample so that a narrow, substantially parallel beam is produced.

11. A method according to claim 10, wherein the lens is a sapphire ball lens.

12. A method according to any preceding claim, which is suitable for measuring the colour of a sample, where each colour has a different absorbance.

13. A method according to claim 12, wherein the sample undergoes a colour change during a chemical reaction.

14. A method according to claim 13, wherein the method is typically carried out after the chemical reaction.

15. A method of monitoring the presence of an analyte on a surface, which method includes:
permitting the surface to contact a reagent so that analyte present on the surface may (chemically) react with the reagent if the analyte is present;
directing a first wavelength along an optical path through a portion of the reagent and measuring the absorbance of the sample so as to obtain a reference absorbance;
directing a second beam of light having a second wavelength substantially along the optical path and measuring the absorbance so as to obtain a measured sample absorbance; and analysing the reference absorbance and the measured sample absorbance to obtain a true sample absorbance which provides an indication the amount of analyte present on the sample which has reacted with the reagent.

16. A method according to claim 15, wherein the indication of the amount of analyte present may be presented in relative colour units.

17. A method according to claim 15 or 16, wherein the analyte is a protein and the reagent comprises a mixture of BCA and copper sulphate.

18. A method according to claim 17, wherein the reagent is a mixture of A(1% disodium-2,2′-bisinchonate, 2% Na₂CO₃.H₂O, 0.16% sodium tartrate, 0.4% NaOH, 0.95% NaHCO₃ and 95.49% water) and B (deionised aquesus solution containing 4% by weight cupric sulfate) preferably in the ratio 100:2.

19. A method according to any of claims 15 to 18, wherein the first wavelength is set at or above 750 nm in the infra red region where the absorbance is similar for purple, grey and green.

20. A method according to claim 19, wherein the first wavelength is at or about 850 mm.

21. A method according to any of claims 15 to 20, wherein the second wavelength is set at or about 562 nm in the green region (where the peak absorbance occurs).

22. A method of monitoring the absorbance of a sample, which method includes directing a first beam of infra red light along a path length through the sample and measuring the absorbance so as to obtain a reference absorbance;
directing a second beam of green light along the path length through the sample and measuring the absorbance of the second beam in the sample so as to obtain a measured sample absorbance; and
analysing the reference absorbance and the measured sample absorbance so as to obtain a true sample absorbance.

23. Apparatus for monitoring the absorbance of a sample, which apparatus includes:
a first light source arranged to emit light at a first wavelength through the sample along an optical path;
a second light source arranged to emit light at a second wavelength through the sample substantially along the same optical path; and
a detector arranged to measure the absorbance of the light emitted from the first light source and the second light source which has passed through the sample.

24. Apparatus according to claim 23, which further includes light directing means arranged to combine the light from the first light source and the light from the second light source so that the light from both light sources travel substantially along the same path.

25. Apparatus according to claim 24, wherein the light directing means is an optical fibre.

26. Apparatus according to claim 24, wherein the optical fibre has a length about 0.5 to 10 mm.

27. Apparatus according to any of claims 22 to 26, wherein the apparatus further includes a lens arranged to produce a narrow, substantially parallel or near parallel beam of light which will subsequently pass through the sample.

28. Apparatus according to claim 27, wherein the lens is a ball lens, such as a sapphire ball lens.

29. Apparatus according to claim 27 or claim 28, wherein the lens is arranged substantially between the light combing means and the sample.

30. Apparatus according to any of claims 22 to 29, which includes a reflector.

31. Apparatus according to claim 30, wherein the reflector is arranged such that light emitted from the first light source and/or the second light source is reflected by the reflector and the reflected light is directed to the light directing means.

32. Apparatus according to any of claims 22 to 29, wherein the first light source and/or the second light source is preferably an LED set to emit light at the first wavelength and/or the second wavelength.

33. Apparatus according any of claims 22 to 30, wherein the apparatus includes two or more LED's, preferably arranged to emit light at different wavelengths.

34. Apparatus according to any of claims 22 to 31, which is suitable for use in monitoring the colour change in a chemical reaction.

35. Apparatus according to any of claims 22 to 32, which is used for monitoring the colour change of a mixture of copper sulphate and BCA (preferably a mixture of A 1% disodium-2,2′-bisinchonate, 2% Na₂CO₃.H₂O, O.16% sodium tartrate, 0.4% NaOH, 0.95% NaHCO₃ and 95.49% water and B deionised aquesus solution containing 4% by weight cupric sulfate preferably in the ratio 100:2), when the mixture is permitted to contact an analyte, such as a protein.
